(19) United States
(12) Patent Application Publication
(10) Pub. No.: US 2020/0390592 A1
(43) Pub. Date: Dec. 17, 2020

(54) Urine Collection and Release System

(71) Applicant: Joseph Merrill, Grand Junction, CO (US)

(72) Inventor: Joseph Merrill, Grand Junction, CO (US)

(21) Appl. No.: 16/443,606

(22) Filed: Jun. 17, 2019

(51) Int. Cl. A61F 5/451; A61F 5/44

(52) U.S. Cl. CPC A61F 5/451; A61F 5/4405; A61F 5/4408

(57) ABSTRACT

A urine collection system disclosed includes a urine collection orifice in communication with a urine collection tube, a urine collection bladder in comunication with the urine collection tube, and a urine release valve comprising an outlet and an inlet in communication with a urine transfer tube. The urine collection system further includes a urine release tube in communication with the outlet of the urine release valve. The urine collection system further includes a substrate for the urine release valve, the substrate molded to a shape of a human leg. Additionally, a push button control of the urine release valve is included, as well as a spring return arm control of the urine release valve and a control cord. A pinch clip control of urine through the urine transfer tube is disposed inline with the urine transfer.

### SUMMARY OF THE INVENTION

A urine collection system disclosed includes a urine collection orifice in communication with a urine collection tube, a urine collection bladder in comunication with the urine collection tube, and a urine release valve comprising an outlet and an inlet in communication with a urine transfer tube. The urine collection system further includes a urine release tube in communication with the outlet of the urine release valve.

The urine collection system further includes a substrate for the urine release valve, the substrate molded to a shape of a human leg. Additionally, a push button control of the urine release valve is included, as well as a spring return arm control of the urine release valve and a control cord. A pinch clip control of urine through the urine transfer tube is disposed inline with the urine transfer.

A leg strap to hold the urine collection bladder adjacent a leg of a user of the urine collection system and a waist strap for disposing the urine release valve adjacent a body of a user of the urine collection system are also included. A urine collection orifice comprising a receiving cup adapted to a female genitalia and a urine collection orifice comprising a receiving cup adapted to a male genitalia are also included.

The urine collection tube, the urine transfer tube and the urine release tubes comprise a low durometer material, quarter inch tubing. The urine collection bladder is pressurized and the urine collection system are pressurized and portions of the system are gravity fed as well.

Other aspects and advantages of embodiments of the disclosure will become apparent from the following detailed description, taken in conjunction with the accompanying drawings, illustrated by way of example of the principles of the disclosure.

### BRIEF DESCRIPTION OF THE DRAWINGS

FIG. 1 is a depiction of the components of a urine collection and release system in accordance with an embodiment of the present disclosure.

FIG. 2 is a depiction of a male gender implementation of the urine collection and release system in accordance with an embodiment of the present disclosure.

FIG. 3 is a depiction a female gender implementation of the urine collection and release system in accordance with an embodiment of the present disclosure.

FIG. 4 is a depiction of a male urine collection orifice in accordance with an embodiment of the present disclosure.

FIG. 5 is a depiction of a female urine collection orifice in accordance with an embodiment of the present disclosure.

Throughout the description, same reference numbers may be used to identify same or similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

Dimensions detailed herein and in the drawings are intended to be a guide to nominal manufacturing dimensions. The detailed dimensions may vary by plus or minus ten percent taking into account manufacturing restraints and materials for various embodiments as recited, taught and suggested herein. The dimensions therefore are applicable to at least one embodiment but are not meant to be limiting to other embodiments of the disclosure.

The present disclosure is a device for urine storage and retrival on demand. It can be used for medical purposes and to satisfy urinalysis examination over an extended period of time for men and women. The disclosure comprises a mine bladder and a ¼ fuel valve bound to a piece of nominal quarter inch plexiglass curved to the contour of a user's leg. The fuel valve controls flow of urine from the bladder. The urine in the bladder maintains a body temperature until it is released from the bladder via the valve. A custom fuel valve arm controls release of urine from the bladder and is activated from pressure of a user's hand on his penis. As soon as the user lets go, the urine stops flowing through the valve.

Embodiments of the disclosure are activated with just a push button fuel valve attached to a valve inline with the urine bladder for female users. The device is not visible when worn under clothing since it is strapped onto a leg of the user and substrates for the components of the system are molded from plastic and plexiglass.

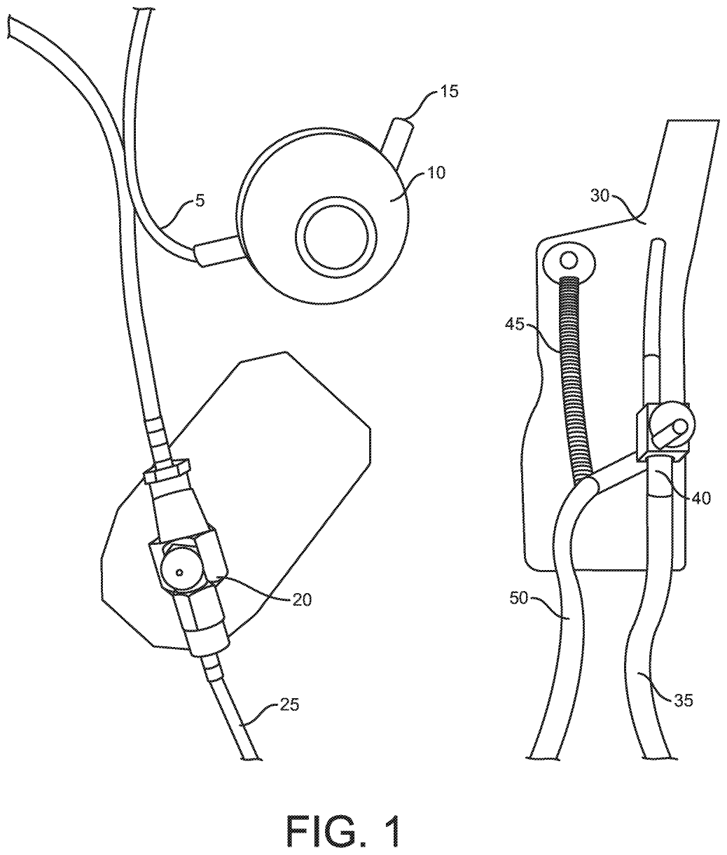

FIG. 1 is a depiction of the components of a urine collection and release system in accordance with an embodiment of the present disclosure. The depiction includes a urine collection tube 5, a urine push button control valve 10, a urine transfer tube 15, a manual urine release valve 20, a urine transfer tube 25 for the manual urine valve 20, a human leg molded plexiglass substrate 30, a urine release tube 35, a arm activate urine release valve 40, a return spring 45 and a urine release control cord 50.

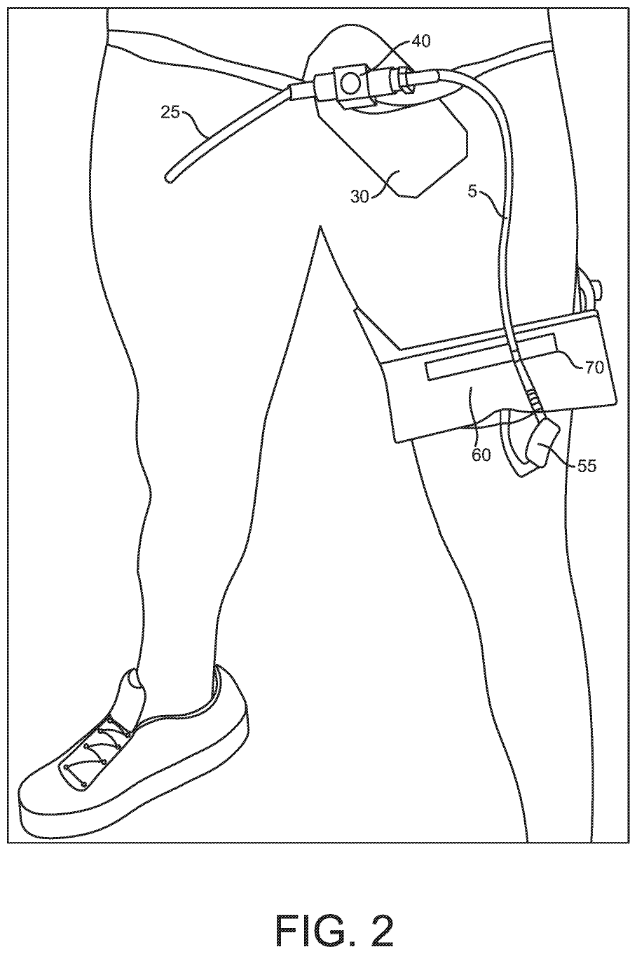

FIG. 2 is a depiction of a male gender implementation of the urine collection and release system in accordance with an embodiment of the present disclosure. The depiction includes the urine transfer tube 5, a urine release tube 25, a human molded plastic substrate 30 for the manual urine release valve 40, a urine collection bladder 60, a pin clip 55 and a urine temperature indicia 70 disposed adjacent the urine collection bladder 60.

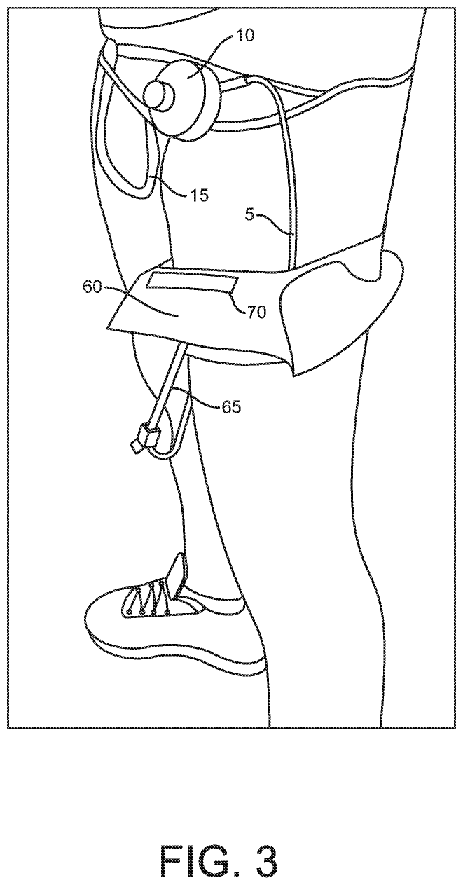

FIG. 3 is a depiction a female gender implementation of the urine collection and release system in accordance with an embodiment of the present disclosure. The depiction includes a urine transfer tube 5, a push button control urine release valve 10, a urine release tube 15, a urine collection bladder 60, a urine collection tube 65 and a temperature indicia 70 disposed adjacent the urine collection bladder 60.

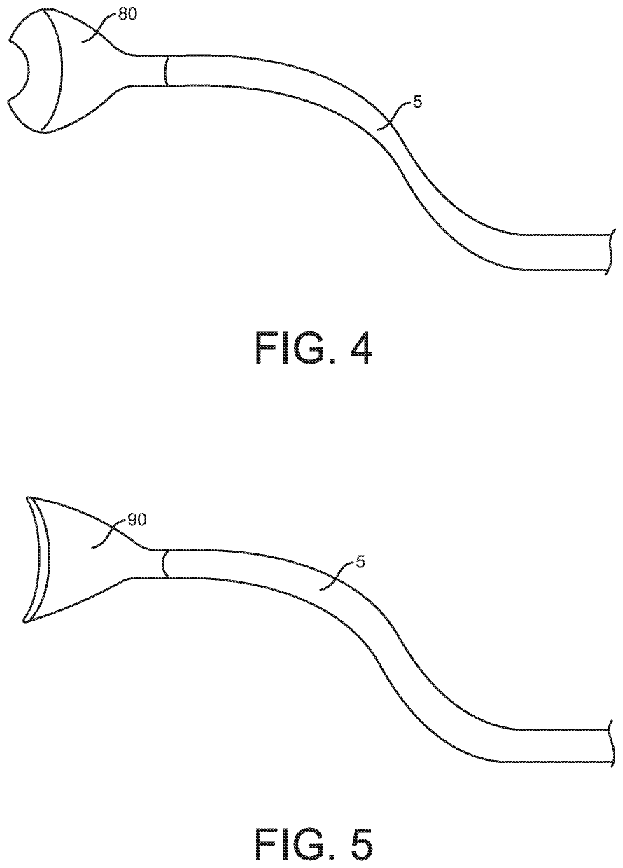

FIG. 4 is a depiction of a male urine collection orifice in accordance with an embodiment of the present disclosure. The depcition includes the receiving cup 80 and the urine collection tube 5. The receiving cup 80 comprises a low durometer silicon which may receive and hold a male penis.

FIG. 5 is a depiction of a female urine collection orifice in accordance with an embodiment of the present disclosure. The depcition includes the receiving cup 90 and the urine collection tube 5. The receiving cup 90 comprises a low durometer silicon which may receive a female vulva.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein.

What is claimed is:

1. A urine collection system, comprising:
a urine collection orifice in communication with a urine collection tube;
a urine collection bladder in communication with the urine collection tube; and
a urine release valve comprising an outlet and an inlet in communication with a urine transfer tube.

2. The urine collection system of claim 1, further comprising a urine release tube in communication with the outlet of the urine release valve.

3. The urine collection system of claim 1, further comprising a substrate for the urine release valve, the substrate molded to a shape of a human leg.

4. The urine collection system of claim 1, further comprising a push button control of the urine release valve.

5. The urine collection system of claim 1, further comprising a spring return arm control of the urine release valve.

6. The urine collection system of claim 1, further comprising a spring return cord control of the urine release valve.

7. The urine collection system of claim 1, further comprising a pinch clip control of urine through the urine transfer tube.

8. The urine collection system of claim 1, further comprising a leg strap for disposing the urine collection bladder adjacent a leg of a user of the urine collection system.

9. The urine collection system of claim 1, further comprising a waist strap for disposing the urine release valve adjacent a body of a user of the urine collection system.

10. The urine collection system of claim 1, wherein the urine collection orifice comprises a receiving cup adapted to a female genitalia.

11. The urine collection system of claim 1, wherein the urine collection orifice comprises a receiving cup adapted to a male genitalia.

12. The urine collection system of claim 1, wherein the urine collection tube and the urine transfer tube comprise a low durometer material quarter inch tubing.

13. The urine collection system of claim 1, wherein the urine collection bladder is pressurized.

14. The urine collection system of claim 1, wherein the system is pressurized.

15. The urine collection system of claim 1, wherein the urine release valve is manually operated.

16. The urine collection system of claim 1, further comprising a temperature indicia disposed adjacent the urine collection bladder.

17. A urine collection method, the method comprising:
receiving urine via a urine collection orifice in communication with a urine collection tube;
collecting urine via a urine collection bladder in communication with the urine collection tube; and
controlling a urine release via a urine release valve comprising an outlet and an inlet in communication with a urine transfer tube.

18. The urine collection method of claim 17, further comprising releasing a collected urine via a urine release tube on the urine release outlet.

19. The urine collection method of claim 17, further comprising controlling a urine release valve via a spring return control arm.

20. A urine collection system, comprising:
a urine collection orifice in communication with a urine collection tube;
a urine collection bladder in communication with the urine collection tube;
a urine release valve comprising an outlet and an inlet in communication with a urine transfer tube; and
a urine release tube in communication with the outlet of the urine release valve.
